(19) United States
(12) Patent Application Publication
(10) Pub. No.: US 2007/0116786 A1
(43) Pub. Date: May 24, 2007

(54) A LIPIDIC EXTRACT FROM LEPIDIUM MEYNII AND ITS EFFECT ON THE LIBIDO

(75) Inventors: Qun Yi Zheng, Wayne, NJ (US); Bo Lin Zheng, Aurora, CO (US); Kan He, River Edge, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NATUREX, Avignon (FR)

(21) Appl. No.: 11/460,142

(22) Filed: Jul. 26, 2006

Related U.S. Application Data
(60) Provisional application No. 60/702,796, filed on Jul. 26, 2005.

(51) Int. Cl. A61K 36/185; A61K 31/56; A61K 31/198; A61K 31/26; A61K 31/165

(52) U.S. Cl. 424/725; 514/171; 514/514; 514/561; 514/617

(57) ABSTRACT

The present invention relates to compositions containing particular components that can be obtained from a plant which can have pharmaceutical applications. More particularly, the plant genus is Lepidium and the composition may contain in the range of between about 0.3% and 0.7% of benzyl isothiocyanate, b) between about 0.06% and about 0.02% of Lepidium sterol component, c) between about 1% and about 2% of a Lepidium fatty acid component, and d) about 0.006% to 0.6% or more total macamide/macaenes component as standardized with excipients.

### BACKGROUND OF THE INVENTION

1. Field of the Invention

The present invention relates to compositions containing particular components that can be obtained from a plant which can have pharmaceutical applications. More particularly, the plant genus is Lepidium.

2. Description of the State of the Art

In the U.S., up to 30 million men are assumed to suffer some degree of erectile dysfunction (Morgentaler, 1999). At least 10 million American men are thought to have impotence. It is estimated that about 70-80% of impotence cases are caused by medical problems, such as hypogonadism, atherosclerosis, hypertension, diabetes mellitus, depression and other psychological illness. Most of impotence cases are treated with Viagra® or other medicines. This treatment is focusing on the physiological mechanics to achieve and maintain erection and do little or nothing to enhance the sexual desire or libido of men suffering erectile dysfunction. Therefore, it is necessary to treat sexual performance disorder in a manner to overcome both the physiological and psychological problems associated with the disorder.

Lepidium meyenii (Brassicaceae), known as Maca or Peruvian ginseng, is a perennial crop of Peru with a long history. Having a fleshy, edible, tuberous root macca was domesticated at least 2000 years ago in the Andean Mountains at an altitude more than 10,000 feet (Leon, 1964). Another species is Lepidium peruvianum. The area where Maca is grown is believed to be the world's worst farmland, with intense sunlight, violent winds, and a temperature that can reach 10° C. to 18° C. below freezing at night. Few other crops can survive in such a harsh environment.

For centuries, the Andean Indians have utilized Maca as a food and for its pharmacological properties; for example to enhance fertility. (See Leon, J., Economic Botany, 18:122-127(1964)). Maca has also been used to treat chronic fatigue. (Steinberg, P., Phil Steinberg's Cat's Claw News, Vol. 1, Issue 2, July/August (1995). As a food source, Maca displays a high nutritional value and is rich in sugars, protein, starches and minerals. Medicinally, it has been used to enhance fertility, a property that tends to be reduced at high altitudes, both in humans and livestock (Johns, 1981).

Even though a highly valued plant of the Andean Indians, little was known about Maca in the outside world. Since the area where Maca is grown had very few scientific travelers, it was not mentioned in many publications, not even in some that were dedicated to special works on the food plants of Peru (Leon, 1964).

In recent years, however, the popularity and the demand of this crop have steadily increased due to its many medicinal properties. Today, Maca is used as a dietary supplement to increase fertility, improve male impotency and as an aphrodisiac for both men and women. With the increasing interest in Maca, many compounds have been identified since 2000. Macaenes and macamides are reported as the major constituents isolated from Maca (Zheng et al., 2000; Muhammad et al., 2002; Zhao et al., 2005). Other chemical structures including alkaloids (Piacente et al., 2002; Cui et al., 2003) and glucosinolates (Dini et al., 2002; Sonia et al., 2002) were also reported. Maca's fertility enhancing reputation has been studied by scientists as early as 1961 (Chacon, 1961). The study, conducted as a dissertation work of a Peru scholar, showed an increased fertility in rats after feeding them with Maca. However, the results were far from satisfactory. Recent in vivo studies have demonstrated that Maca extract possesses the properties of improving animal sexual performance (Zheng et al., 2000, 2001, 2002, 2003; Cicero et al., 2001; Cicero et al., 2002) and preventing high altitude-induced spermatogenic disruption (Gonzales et al., 2004). A clinical trial indicated that Maca extract improved sexual desire without affecting serum reproductive hormone levels (Gonzales et al., 2002). Maca has also been found to improve physical and mental health, enhance mental clarity, and increase energy, stamina, and endurance for athletes (Zheng et al., 2002).

### BRIEF SUMMARY OF THE INVENTION

One embodiment of the present invention utilizes a Lepidium-derived pharmaceutical composition to treat sexual dysfunction.

Another aspect of the present invention relates to an isolated, Lepidium-derived composition that can contain an aqueous component and a component, designated a Lepidium amino acid component, having amino acids that can be isolated from Lepidium plant material. Typically, the Lepidium amino acid component has about 70% or more proline, 5% or more glutamic acid, and 5% or more valine. In certain embodiments, the composition has about 0.3% benzyl isothiocyanate and about 0.5% of a component, called a macamide component, having amides of fatty acids that can be isolated from Lepidium material. In another embodiment, the composition is combined with one or more pharmaceutically acceptable excipients.

In another aspect, the present invention relates to a composition having about 0.3% or more benzyl isothiocyanate, about 0.3% or more of a macamide component, about 1% or more of fatty acids that can be isolated from Lepidium plant material (a Lepidium fatty acid component), and about 0.15% of sterols that can be isolated from Lepidium plant material (a Lepidium sterol component).

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF THE INVENTION

As used herein, the following terms have the following meanings.
Alcohol: refers to a lower aliphatic alcohol having from one to six carbon atoms.
Aqueous component: refers to that part or portion of a composition that is made-up of one or more aqueous solvents.
Aqueous solvent: refers to water or a single phase having an organic solvent that is miscible with water. Examples of miscible organic solvents include but are not limited to methanol, ethanol, isopropanol, n-propanol, acetone, and acetonitrile. Other miscible organic solvents are known to the skilled artisan.
Benzyl isothiocyanate: includes benzyl isothiocyanate and its methoxy derivatives.
Lepidium amino acid component: refers to that part or portion of a composition that is made-up of amino acids that can be isolated from Lepidium plant material.
Lepidium composition: refers to a composition having at least one of a Lepidium amino acid component, a Lepidium fatty acid component, a Lepidium polysaccharide component, or a macamide component.
Lepidium fatty acid component: refers to that part or portion of a composition that is made-up of fatty acids that can be isolated from Lepidium plant material.
Lepidium plant material: refers to plant matter from any part of a plant of the genus Lepidium. Examples of Lepidium plant matter include, but are not limited to, matter from Lepidium meyenii and Lepidium peruvianum.
Lepidium polysaccharide component: refers to that part or portion of a composition that is made-up of polysaccharides that can be isolated from Lepidium plant material.
Lepidium sterol component: refers to that part or portion of a composition that is made-up of sterols that can be isolated from Lepidium plant material.
Macamide: refers to amides and N-substituted amides of fatty acids that can be isolated from Lepidium plant material.
   Percent (%): Unless otherwise limited or modified, percents and percentages described herein are on a weight basis. The chemical composition of plant material from a particular plant species varies with, for example, the conditions under which the plant is grown (for example soil and climate). A particular compound or mixture of compounds can exhibit pharmacological efficacy over a readily ascertainable range of composition and dosage. Therefore, it is understood that the percentages recited throughout are meant to include such variations outside the stated percentage or percentage ranges as would be anticipated by the skilled artisan.

An embodiment of the present invention is the use and effect of a Lepidium-derived pharmaceutical composition to treat erectile dysfunction. An embodiment of the present invention utilizes MacaPure® (further described below) as an embodiment of a Lepidium derived pharmaceutical composition. A clinical study was conducted to assess the effect of MacaPure® on erectile function and sexual desire in subjects with a clinical diagnosis of mild or mild-to-moderate erectile dysfunction. The study was a 10-week randomized, double-blind, placebo-controlled, and crossover clinical trial. Erectile function and sexual desire or libido were assessed using the International Index of Erectile Function (IIEF) questionnaire. The questionnaire consisted of 15 multiple-choice questions. For every question, five to six possible answers were given. The answer with higher scores indicated more favorable outcomes.

The 15 questions were divided into 5 domains:

Domain 1, erectile function: Questions 1+2+3+4+5+15

Domain 2, orgasmic function:Questions 9+10

Domain 3, sexual desire: Questions 11+12

Domain 4, intercourse satisfaction: Questions 6+7+8

Domain 5, overall satisfaction: Questions 13+14

In addition to the IIEF questionnaire, the Sexual Encounter Profiles (SEP) diary, an event log for recording sexual activity and quality of the erection, was completed throughout supplementation.

Subjects and Study Procedures

A total of 32 subjects with a clinical diagnosis of mild or mild-to-moderate erectile dysfunction were included in the study. The subjects were randomized over two groups (I: MacaPure® followed by placebo; II: Placebo followed by MacaPure®) and received a supplement containing 900 mg Maca extract (MacaPure®) per day in one treatment period, and a Placebo (P) supplement in the other period. During an initial supplementation period of four weeks (period A), the patients received either a MacaPure® or a placebo supplement. After a washout period of two weeks, patients receiving Maca received placebo for four weeks and vice versa (period B). During the study, Erectile function and libido were measured using the IIEF questionnaire (Q) and SEP diary. The protocol of the study is listed below.

[image]

V1: screening visit; V2: randomization visit (t=0weeks); V3: visit after 2 weeks (after two weeks supplementation in period A); V4: visit after 4 weeks (end of period B); Date phone call: phone call after 6 weeks (end of wash out period; start of period B); V5: visit after 8 weeks (after two weeks supplementation in period B); V6: visit after 10 weeks (end of period B). At the end of each treatment period, safety measurements (S) such as blood parameters (creatinine, gamma GT, glucose, LDH, AST, ALT), blood pressure, and pulse rate was performed. Also plasma total testosterone was measured.

Primary and Secondary Efficacy Variables

The primary outcome variable of the study was within-subject difference in erectile function between 4 weeks of MacaPure® supplementation and 4 weeks of placebo supplementation. Erectile function was measured by the IIEF questionnaire, including IIEF Domain 1 (as measured by the sum of questions 1+2+3+4+5+15), and the questions 3 and 4 as primary efficacy parameters.

Secondary efficacy parameters included the difference in other domains of the IIEF between 4 weeks of MacaPure® supplementation and 4 weeks of placebo supplementation Domain 2 (Orgasmic function, questions 9+10); Domain 3 (Sexual desire, questions 11+12); Domain 4 (Intercourse satisfaction, questions 6+7+8); Domain 5 (Overall satisfaction, questions 13+14). In addition, within-subject differences in individual questions and in SEP recordings were assessed.

Material

MacaPure® was manufactured by PureWorld Botanicals, Inc., South Hackensack, N.J., (which has been acquired by Naturex of Paris, France). MacaPure® is a standardized product which contains 0%-10% total macamides/macaenes. The structures of macaenes and macamides are 9,16-dioxo-10E,12E,14E-octadecatrienoic acid, 9-Oxo-10E,12E,15E-octadecatrienoic acid, 16-hydroxy-9-oxo-10E,12E,14E-octadecatrienoic acid, 2′3′-dihydroxypropyl hexadecanoicate, 9-Oxo-10E,12E-octadecadienoic acid ethylester, N-benzyloctanamide, N-benzyl-16-hydroxy-9-oxo-10E,12E,14E-octadecatrienamide, N-benzyl-9,16-dioxo-10E,12E,14E-octadecatrienamide, N-benzyl-hexadecanamide, N-benzyl-9-oxo-10E,12E-octadecadienamide, N-benzylformamide, N-benzyl-9-oxo-10E,12E,15E-octadecatrienamide, N-benzyl-9Z, 12Z, 15Z-octadecatrienarnide, and N-benzyl-9Z-octadecaenamide. MacaPure® also containes imidazole alkaloids including lepidiline A and B, sterols including β-sitersterol and β-sitersterol-3-O-β-D-glycopyranoside, fatty acids including palmitic acid, oleic acid, linoleic acid, and linolenic acid, sugars including glucose, fructose, sucrose, maltose, and lactose, mannitol, polysaccharides, protein, and minerals including potassium, magnesium, calcium, sodium, phosphorous, copper, zinc, and iron.

The actual amount per daily dose at 3 tablets of MacaPure® contains 5.4 mg of macamides/macaenes (from 900 mg Maca standardized product which contains 0.6% macamides/macaenes), 1151 mg of Endurance Plus Excipient (microcrystalline cellulose, calcium carbonate, elemental calcium), 1380 mg of Prosolv 90 (microcrystalline cellulose, colloidal silicon dioxide), 979 mg of Dicalcium phosphate (Ca 23.4%; P 18%), Total weight per 3 tablets(including excipients) of MacaPure® is 4410 mg. The actual amount per daily 3 tablets of placebo contains 936 mg of Prosolv 90, 979 mg of Dicalcium phosphate only. Total weight per daily dose at 3 tablets of placebo is 5235 mg.

A MacaPure® tablet may contain in the range of between about 0.3% and 0.7% of benzyl isothiocyanate, b) between about 0.6% and about 0.2% of Lepidium sterol component, c) between about 1% and about 2% of a Lepidium fatty acid component, and d) about 0.006% to 0.6% or more total macamide/macaenes component as standardized with exipients.

Results

Results of MacaPures® versus Placebo are shown in Table 1. No signficant differences in IIEF Domain 1 (Erectile function), IIEF question 3 (Ability to penetrate), and IIEF question 4 (Ability to maintain erection) were detected between the periods of MacaPure® and placebo supplementation.

TABLE 1
Erectile function, as measured by the IIEF questionnaire domain 1,
question 3, and question 4, after 4 weeks of MacaPure ® or
placebo supplementation.^{a}

| | MacaPure ® | Placebo | |
|---|---|---|---|
| | (n = 32) | (n = 32) | P-value |
| IIEF D1 (Erectile function)^{c} | 19.8 ± 1.2 | 19.6 ± 1.1 | .815 |
| IIEF Q3 (Ability to penetrate)^{d} | 3.5 ± 0.2 | 3.8 ± 0.2 | .347 |
| IIEF Q4 (Ability to maintain | 3.1 ± 0.3 | 3.1 ± 0.3 | .000 |
| erection)^{d} | | | |

^{a}Mean ± SEM
^{b}No significant differences (Two-sample Wilcoxon rank-sum test with correction for ties)
^{c}Scoring range: 1 (low performance) to 30 (high performance)
^{d}Scoring range: 0 (low performance) to 5 (high performance)

For the experiment of MacaPure® versus Placebo, study results regarding the scores to the Sexual Encounter Profile diary are shown in Table 2. No statistically significant differences between MacaPure® and placebo supplementation were detected.

TABLE 2
Scores to the Sexual Encounter Profile diary during 4 weeks of
MacaPure ® or placebo supplementation.

| | MacaPure ® | Placebo | |
|---|---|---|---|
| | (n = 31) | (n = 31) | P-value^{c} |
| Number of sexual attempts | 4.8 ± 0.4 | 4.3 ± 0.4 | 0.135 |
| per week^{a} | | | |
| % succesful erections^{b} | 100 (20-100) | 100 (25-100) | 0.514 |
| % succesful insertions^{b} | 100 (0-100) | 100 (0-100) | 0.357 |
| % erections lasting long | 63 (0-100) | 50 (0-100) | 0.143 |
| enough^{b} | | | |
| % erections satisfactory | 11 (0-100) | 0 (0-100) | 0.493 |
| regarding hardness^{b} | | | |
| % overall satisfaction^{b} | 6 (0-100) | 0 (0-100) | 0.333 |

^{a}Mean ± SEM
^{b}Median (range)
^{c}No statistically significant differences were detected (Two-sample Wilcoxon rank-sum test with correction for ties)

For the experiment of MacaPure® versus Placebo, study results regarding the separate IIEF questions and domains are shown in Table 3. The rating of confidence to get and keep an erection (Question 15) was significantly higher during MacaPure® than during placebo supplementation (P=0.031).

TABLE 3
Scores to the separate questions and domains of the IIEF questionnaire
after 4 weeks of MacaPure ® or placebo supplementation.^{a,b}

| | MacaPure ® | Placebo | |
|---|---|---|---|
| | (n = 32) | (n = 32) | P-value |
| Q1 (“how often were you able to get an erection?”) | 4.1 ± 0.2 | 4.1 ± 0.2 | 0.879 |
| Q2 (“how often were your erections hard enough for penetration?”) | 3.4 ± 0.2 | 3.7 ± 0.2 | 0.208 |
| Q5 (“how difficult was it to maintain your erection?”) | 2.9 ± 0.2 | 2.6 ± 0.2 | 0.191 |
| Q6 (“how many times have you attempted sexual intercourse?”) | 3.0 ± 0.2 | 2.7 ± 0.2 | 0.148 |
| Q7 (“how often was the sexual intercourse satisfactory for you?”) | 2.9 ± 0.3 | 2.9 ± 0.3 | 0.919 |
| Q8 (“how much have you enjoyed sexual intercourse?”) | 3.0 ± 0.2 | 3.2 ± 0.2 | 0.261 |
| Q9 (“how often did you ejeculate?”) | 3.8 ± 0.3 | 3.8 ± 0.2 | 0.946 |
| Q10 (“how often did you have the feeling of orgasm?”) | 3.6 ± 0.3 | 3.4 ± 0.3 | 0.483 |
| Q11 (“how often have you felt sexual desire?”) | 3.6 ± 0.2 | 3.8 ± 0.2 | 0.372 |
| Q12 (“how would you rate your level of sexual desire?”) | 3.2 ± 0.2 | 3.4 ± 0.2 | 0.397 |
| Q13 (“how satisfied have you been with your overall sex life?”) | 2.5 ± 0.2 | 2.5 ± 0.2 | 0.885 |
| Q14 (“how satisfied have you been with your sexual relationship?”) | 3.3 ± 0.2 | 3.0 ± 0.3 | 0.422 |
| Q15 (“how do you rate your confidence that you can get and keep your | 2.8 ± 0.2 | 2.3 ± 0.2 | 0.031 |
| erection?”) | | | |
| Domain 2 (Orgasmic function) | 7.4 ± 0.5 | 7.3 ± 0.5 | 0.980 |
| Domain 3 (Sexual desire) | 6.8 ± 0.4 | 7.2 ± 0.4 | 0.297 |
| Domain 4 (Intercourse satisfaction) | 8.9 ± 0.5 | 8.8 ± 0.5 | 0.873 |
| Domain 5 (Overall satisfaction) | 5.8 ± 0.4 | 5.5 ± 0.5 | 0.538 |

^{a}Mean ± SEM
^{b}No statistically significant differences were detected (Two-sample Wilcoxon rank-sum test with correction for ties)

Efficacy Conclusions

From the present clinical trial, MacaPure® did not have statistically significant effects on the score to IIEF Domain 1 (Erectile function), to IIEF question 3 (Ability to penetrate), or to IIEF question 4 (Ability to maintain erection). For the secondary outcome parameters, however, MacaPure® showed to some extent an effect on the sexual desire from the number of sexual attempts per week (as measured by the Sexual Encounter Profile diary as well as by the IIEF question 6). The rating of confidence to get and keep an erection (Question 15) was significantly higher during during MacaPure® supplementation than during placebo supplementation (P<0.05).

Results of the Plasma Testosterone Levels

For the experiment of MacaPure® versus Placebo, study results regarding plasma testosterone levels are shown in Table 4. No statistically significant differences in testosterone levels between MacaPure® and placebo supplementation were detected.

TABLE 4
Plasma testosterone levels after 4 weeks of MacaPure ® and placebo
supplementation (nmol/L).^{a,b}

| Plasma testosterone | | | |
|---|---|---|---|
| (nmol/L) | MacaPure ® | Placebo | P-value |
| Experiment (n = 32) | 14.3 ± 1.2 | 13.5 ± 0.9 | ns |

^{a}Mean ± SEM
^{b}No statistically significant differences were detected (Two-sample Wilcoxon rank-sum test with correction for ties)

Safety Evaluation

Adverse Events

During the 10 week trial period, adverse events were documented by the researcher at Visit 2 (week 0), Visit 3 (Week 2), Visit 4 (Week 4), Visit 5 (Week 8) and visit 6 (week 10). Subjects were asked to record start date, stop date, duration of event in days, intensity of the event, action that was taken and the outcome. The researchers also documented the relationship with the supplement.

In Table 5, adverse events and number of subjects suffering from the adverse event are shown. Study results of subjects suffering from an adverse event have been compared with the mean study results of the complete study group. Results of this analysis showed that the occurrence of the adverse events in MacaPure® does not appear to have affected study outcome, such as the number of attempts and sexual desire.

TABLE 5
Number of subjects suffering from a certain adverse effect during
supplementation of MacaPure ® and Placebo.

| | MacaPure ® | Placebo | Total |
|---|---|---|---|
| Adverse event | (n = 32) | (n = 64) | (n = 64) |
| Common Cold | n = 4 | n = 1 | n = 6 |
| Upper respiratory | | n = 3 | n = 4 |
| infection | | | |
| Insomnia | | n = 1 | n = 3 |
| Nausea | n = 1 | | n = 1 |
| Boils | | n = 1 | n = 2 |
| Excessive urination | | | n = 1 |
| Total | n = 5 | n = 6 | n = 17 |

Clinical Laboratory Evaluation

As safety endpoints, within-subject changes in liver function (plasma gamma-GT, LD, AST, and ALT), kidney function (plasma creatine) and plasma glucose were determined.

For experiment of MacaPure® versus Placebo, blood laboratory values are shown in Table 6. No statistically significant differences in blood values between MacaPure® and placebo supplementation were detected.

TABLE 6
Blood laboratory values after 4 weeks of MacaPure ® or placebo
supplementation.^{a}

| | MacaPure ® | Placebo | |
|---|---|---|---|
| | (n = 32) | (n = 32) | P-value^{b} |
| Creatinine (umol/L) | 101.1 ± 2.4 | 99.2 ± 2.7 | 0.725 |
| Glucose (mmol/L) | 6.4 ± 0.6 | 6.8 ± 0.9 | 0.103 |
| Alanine transaminase (ALT; U/L) | 29.3 ± 2.1 | 29.6 ± 2.0 | 0.211 |
| Aspartate transaminase (AST; U/L) | 19.8 ± 1.1 | 20.2 ± 0.9 | 0.674 |
| Gamma glutamyl transferase | 37.5 ± 4.6 | 35.4 ± 3.0 | 0.079 |
| (GGT; U/L) | | | |
| LDH, total (U/L) | 152 ± 6 | 149 ± 5 | 0.675 |

^{a}Mean ± SEM
^{b}No statistically significant differences were detected (Two-sample Wilcoxon rank-sum test with correction for ties)

Vital Signs, Physical Findings and Other Observations Related to Safety

As additional safety endpoints, within-subject changes in vital signs (systolic and diastolic blood pressure, and pulse) were determined.

For experiment of MacaPure® versus Placebo, vital signs are reported in Table 7. No statistically significant differences in blood pressure or pulse between MacaPure® and placebo supplementation were detected.

TABLE 7
Blood pressure and pulse after 4 weeks of MacaPure ® or placebo
supplementation.^{a}

| | MacaPure ® | Placebo | |
|---|---|---|---|
| | (n = 32) | (n = 32) | P-value^{b} |
| Systolic blood pressure (mmHg) | 125 ± 2 | 125 ± 2 | ns |
| Diastolic blood pressure (mmHg) | 80 ± 1 | 78 ± 1 | ns |
| Pulse (min⁻¹) | 73 ± 2 | 71 ± 2 | ns |

^{a}Mean ± SEM
^{b}ns: Not statistically significant (P > 0.05; Two-sample Wilcoxon rank-sum test with correction for ties)

Safety Conclusions

Supplementation of MacaPure® did not result in the occurrence of major adverse events. The occurrence of insomnia may be related with the supplement, but it is not likely that the other adverse events are related to supplementation of the study product. No clinically significant changes in blood laboratory values or vital signs were found. It is concluded that supplementation of MacaPure® has no undesirable side effects.

Overall Conclusions

Lepidium-derived pharmaceutical compositions such as MacaPure®, a natural derived product, have shown an ability to increase sexual desire or libido. It is well known that low testosterone levels are related to low sexual desire. Therefore, testosterone is often used in both sexual motivation and sexual performance. However, an overdose of testosterone can change the prostate which might be associated with a disorder, such as prostate cancer. Therefore it is not recommended that testosterone be used in a normal man. Based on clinical results, a Lepidium-derived pharmaceutical composition such as MacaPure® can enhance sexual desire without increasing testosterone level. Due to this effect and the safety for human consumption, a Lepidium-derived pharmaceutical composition such as MacaPure® has great potential to be applied to increase libido and to increase the quality of life.

Formulations

It is further contemplated that the Lepidium-derived pharmaceutical compositions of the present invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, and esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

References

Chacon, R.C. Estudiofitoquimico de Lepidium meyenii. Dissertation, Univ., Nac. Mayo de San marcos, Peru. 1961.

Cicero, A.F.G., Bandieri, E., Arletti, R. Lepidium meyenii Walp. improves sexual behaviour in male rats independently from its action on spontaneous locomotor activity. J. Ethnopharmacology 75:225-229, 2001.

Cicero, F. G., Piacente, S., Plaza, A., Sala, E., Arletti, R. and Pizza, C. Hexanic Maca extract improves rat sexual performance more effectively than methanolic and chloroformic Maca extracts. Andrologia 34: 177-179, 2002.

Cui, B. L., Zheng, B. L., He, K., Zheng, Q. Y. Imidazole alkaloids from Lepidium meyenii. J. Nat. Prod. 66, 1101-1103, 2003.

Dini, I., Tenore, G. C., and Dini, A. Glucosinolates from maca (Lepidium meyenii). Biochemical Systematics and Ecology 30: 1087-1090, 2002.

Gonzales, G. F., Córdova, A., Vega, K., Chung, A., Villena, A., Góñez, C. and Castillo, S. Effect of Lepidium meyenii (MACA) on sexual desire and its absent relationship with serum testosterone levels in adult healthy men. Andrologia 34: 367-372, 2002.

Gonzales, G. F., Gasco, ., Córdova, A. Chung, A., Rubio, J. and Villegas, L. Effect of Lepidium meyenii (Maca) on spermatogenesis in male rats acutely exposed to high altitude (4340 m). Journal of Endocrinology 180: 87-95, 2004.

Gonzales, G. F., Córdova, A., Vega, K., Chung, A. Villena, A. and Góñez, C. Effect of Lepidium meyenii (Maca), a root with aphrodisiac and fertility-enhancing properties, on serum reproductive hormone levels in adult healthy men. J. Endocrinology 176: 163-168, 2003.

Johns, T. The añu and the maca. J. Ethnobiology 1: 208-212, 1981.

Leon, J. The “maca” (Lepidium meyenii) a little known food plant of Peru. Economic Botany 18: 122-127, 1964.

Morgentaler, A. Male impotence. Lancet 354 (9191): 1713-8, 1999.

Muhammad, I., Zhao, J., Dunbar, D. C. and Khan, I. A. Constituents of Lepidium meyenii ‘maca’. Phytochemistry 59: 105-110, 2002.

Piacente, S., Carbone, V., Plaza, A., Zampelli, A. and Pizza, C. Investigation of the tuber constituents of maca (Lepidium meyenii Walp.). J. Agric. Food Chem. 50, 5621-5625, 2002.

Zhao, J., Muhammad, I., Dunbar, D. C., Mustafa, J. and Khan, I. A. New alkamides from maca (Lepidium meyenii). J. Agric. Food Chem. 53: 696-693, 2005.

Zheng, B., He, K., Kim, C. H., Rogers, L., Shao, Y., Haung, Z. Y., Lu, Y., Yan, S. J., Qien, L. C., Zheng, Q. Z. Effect of a lipidic extract from Lepidium meyenii on sexual behavior in mice and rats. Urology 55: 598-602, 2000.

Zheng, B. L., He, K., Hwang, Z. Y., Lu, Y., Yan, S. J., Kim, C. H., Zheng, Q. Y. Effect of aqueous extract from Lepidium meyenii on mouse behavior in forced swimming test, In Quality Management of Nutraceuticals (Ho, C. T. and Zheng, Q. Y., Eds.) pp. 90-100, Oxford University Press: American Chemical Society, Washington, D.C., 2002.

Zheng, B. L., He, K., Shao, Y. and Zheng, Q. Y. Compositions and methods for their preparation from Lepidium meyenii roots. U.S. Pat. No. 6,552,206 B1, Apr. 22, 2003.

Zheng, B. L., Kim, C. H., Wolthoff, S., He, K., Rogers, L. L., Shao, Y. and Zheng, Q. Y. Extract of Lepidium meyenii roots for pharmaceutical applications. U.S. Pat. No. 6,267,995 B1, Jul. 31, 2001.

Zheng, B. L., Kim, C. H., Wolthoff, S. W., He, K., Rogers, L. L., Shao, Y. and Zheng, Q. Y. Treatment of sexual dysfunction with an extract of Lepidium meyenii roots. U.S. Pat. No. 6,428,824 B1, Aug. 6, 2002.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A Lepidium-derived pharmaceutical composition comprising:
an aqueous component, wherein said aqueous component includes ethanol; and
a Lepidium amino acid component;

2. The composition of claim 1 wherein said Lepidium amino acid component comprises a amino acid isolated from Lepidium plant material;

3. The composition of claim 1 wherein said Lepidium amino acid component comprises about 70% or more proline, about 5% or more glutamic acid, and about 5% or more valine;

4. The composition of claim 1 wherein said composition further comprises about 0.3% benzyl isothiocyanate and a macamide component.

5. The composition of claim 4 wherein said macamide component comprises amides of fatty acids isolated from said Lepidium plant material.

6. The composition of claim 5 wherein said composition is further combined with one or more pharmaceutically acceptable excipients.

7. A Lepidium-derived pharmaceutical composition comprising:
an aqueous component; and
a Lepidium amino acid component;
wherein said Lepidium amino acid component comprises a amino acid isolated from Lepidium plant material;
about 0.3% or more benzyl isothiocyanate;
about 0.3% or more of a macamide component;
about 1% or more of fatty acids; and
about 0.15% or more of sterols.

8. The composition of claim 7 wherein said Lepidium amino acid component comprises about 70% or more proline, about 5% or more glutamic acid, and about 5% or more valine.

9. The composition of claim 7 wherein said macamide component comprises amides of fatty acids isolated from said Lepidium plant material.

10. The composition of claim 7 wherein said composition is further combined with one or more pharmaceutically acceptable excipients.

11. A Lepidium-derived pharmaceutical composition comprising:
an aqueous component; and
a Lepidium amino acid component;
wherein said Lepidium amino acid component comprises a amino acid isolated from Lepidium plant material;
between about 0.3% and about 0.7% of benzyl isothiocyanate,
between about 0.6% and about 0.2% of a Lepidium sterol component;
between about 1% and about 2% of a Lepidium fatty acid component; and
about 0.006% and about 0.6% or more total macamide/macaenes component as standardized with excipients.

12. A method of treating sexual dysfunction comprising the administration of a therapeutic dose of the composition of claim 1.

13. A method of treating sexual dysfunction comprising the administration of a therapeutic dose of the composition of claim 7.

14. The method of claim 12 wherein said sexual dysfunction to be treated is erectile function.

15. The method of claim 12 wherein said sexual dysfunction to be treated is libido.

16. The method of claim 12 wherein said sexual dysfunction to be treated is orgasmic function.

17. The method of claim 12 wherein said sexual dysfunction to be treated is sexual desire.

18. The method of claim 12 wherein said sexual dysfunction to be treated is intercourse satisfaction.

19. The method of claim 12 wherein said sexual dysfunction to be treated is overall sexual satisfaction.

20. A method of treating said sexual dysfunction of claim 12 comprising the administration of a daily therapeutic dose of a Lepidium-derived pharmaceutical composition comprising:
about 5.4 mg of macamides/macaenes;
about 1151 mg of a microcrystalline cellulose, calcium carbonate, elemental calcium component;
about 1380 mg of a microcrystalline cellulose, colloidal silicon dioxide component; and
about 979 mg of Dicalcium phosphate component.
